(19) United States
(12) Patent Application Publication
(10) Pub. No.: US 2012/0316532 A1
(43) Pub. Date: Dec. 13, 2012

(54) Disposable Absorbent Article With Topsheet Having A Continuous, Bonded Pattern

(76) Inventor: Sarah Ann MCCORMICK, Wyoming, OH (US)

(21) Appl. No.: 13/470,394

(22) Filed: May 14, 2012

Related U.S. Application Data
(60) Provisional application No. 61/496,560, filed on Jun. 13, 2011.

(51) Int. Cl. A61F 13/513; A61L 15/24

(52) U.S. Cl. 604/372; 604/385.01

(57) ABSTRACT

Topsheets characterized by having a certain basis weight, activating the topsheet prior to making a continuous bonded pattern on it, where multiple continuous patterns are optionally arranged along the longitudinal axis of the topsheet. The topsheets are optionally, further characterized with pre-bonding, embossing, opacity, and/or coloring.

### CROSS REFERENCE TO RELATED APPLICATION

This application claims the benefit of U.S. Provisional Application No. 61/496,560, filed Jun. 13, 2011, which is herein incorporated by reference in its entirety.

### FIELD OF THE INVENTION

The present disclosure relates to disposable absorbent articles having a topsheet, backsheet, acquisition layer, and absorbent core, and more particularly, to disposable absorbent articles having a topsheet having a continuous bonded pattern.

### BACKGROUND OF THE INVENTION

Disposable absorbent articles for receiving and retaining bodily discharges such as urine or feces are known. Examples of these include disposable diapers, training pants, feminine sanitary napkins, and adult incontinence articles. Typically, such articles comprise a liquid pervious topsheet that faces the wearer's body, a liquid impervious backsheet that faces the wearer's clothing and an absorbent core interposed between the liquid pervious topsheet and the backsheet.

Since being introduce to the market place, disposable absorbent articles continue to improve regarding comfort, fit and functionalities. Topsheets of such absorbent articles may function to absorb and/or contain the discharged materials and also to isolate bodily exudates from the wearer's skin and from the wearer's garments and bed clothing. In some instances, these substrates are substantially smooth, flat and aesthetically unappealing, to begin with. As other levers of functionality of the components of the absorbent article are changed to improve, e.g. absorbency, fit, and reduced cost, the performance and/or appearance of the topsheet is sometimes affected negatively. Efforts have been made to modify these substrates in order to provide them with a particular appearance. For example, such substrates may be modified to exhibit a softer, quilted, and/or cloth-like appearance. In some examples, these substrates may be modified to include an interior design signal to communicate to a caregiver that a relatively thin absorbent article provides adequate absorbency. As such, nonwoven fabrics and/or plastic films are sometimes modified to provide a physical or actual three-dimensional pattern. Non-limiting examples of known methods which provide an actual three-dimensional appearance to a substrate include embossing. While absorption capacity is quite important for diapers, consumers also appreciate products having a soft feel to the touch, especially in regions of the article which directly contact the skin of the wearer, such as a diaper's topsheet. Attempts have been made to increase the softness of the topsheet, such as by selecting a particular fiber type or by reducing fibers bonding within the material, e.g. a nonwoven material, used for the manufacturing of the topsheet. Typically, fibers' bonding imparts strength to the material. Strength is needed to provide resistance to tearing during use of the absorbent article and also during the manufacturing process. Unfortunately, when steps are undertaken to increase the softness of the topsheet, such as decreasing the bonding area to increase softness, strength may be adversely affected. As such, there is a need for improved absorbent articles having topsheets that exhibit excellent performance and are aesthetically pleasing.

### SUMMARY OF THE INVENTION

Disclosed are topsheets characterized by having a certain basis weight, activating the topsheet prior to making a continuous bonded pattern on it, where multiple continuous patterns are optionally arranged along the longitudinal axis of the topsheet. The topsheets are optionally, further characterized with pre-bonding, embossing, opacity, and/or coloring.

### BRIEF DESCRIPTION OF THE DRAWINGS

FIG. 1 is a plan view of a diaper.

FIG. 2 is a cross sectional view of the diaper shown in FIG. 1 taken along the sectional line 2-2 of FIG. 1.

FIG. 3 is a partial cross sectional view of an absorbent core layer.

FIG. 4 is a partial cross sectional view of an absorbent core layer.

FIG. 5A is a partial sectional view of an absorbent core comprising a combination of first and second absorbent core layers described herein.

FIG. 5B is a partial sectional view of an absorbent core comprising a combination of the first and second absorbent core layers described herein.

FIG. 6 is a plan view of a diaper topsheet comprising a continuous bonded pattern.

FIG. 7 is a plan view of a diaper topsheet comprising a continuous bonded pattern.

FIG. 8 is a plan view of a diaper topsheet comprising a continuous bonded pattern.

FIG. 9 is a plan view of a diaper topsheet comprising a continuous bonded pattern.

### DETAILED DESCRIPTION OF THE INVENTION

The following term explanations may be useful in understanding the present disclosure:

“Absorbent article” refers to devices that absorb and contain body exudates, and, more specifically, refers to devices that are placed against or in proximity to the body of the wearer to absorb and contain the various exudates discharged from the body. Absorbent articles may include diapers, training pants, adult incontinence undergarments, feminine hygiene products, breast pads, care mats, bibs, wound dressing products, and the like. As used herein, the term “body fluids” or “body exudates” includes, but is not limited to, urine, blood, vaginal discharges, breast milk, sweat and fecal matter.

“Absorbent core” means a structure that may be disposed between a topsheet and backsheet of an absorbent article for absorbing and containing liquid received by the absorbent article and may comprise one or more substrates, absorbent polymer material disposed on the one or more substrates, and a thermoplastic composition on the absorbent particulate polymer material and at least a portion of the one or more substrates for immobilizing the absorbent particulate polymer material on the one or more substrates. In a multilayer absorbent core, the absorbent core may also include a cover layer. The one or more substrates and the cover layer may comprise a nonwoven. Further, the absorbent core may be substantially cellulose free. The absorbent core does not include an acquisition system, a topsheet, or a backsheet of the absorbent article. In a certain embodiment, the absorbent core would consist essentially of the one or more substrates, the absorbent polymer material, the thermoplastic composition, and optionally the cover layer.

“Absorbent polymer material,” “absorbent gelling material,” “AGM,” “superabsorbent,” and “superabsorbent material” are used herein interchangeably and refer to cross linked polymeric materials that can absorb at least 5 times their weight of an aqueous 0.9% saline solution as measured using the Centrifuge Retention Capacity test (Edana 441.2-01).

“Absorbent particulate polymer material” is used herein to refer to an absorbent polymer material which is in particulate form so as to be flowable in the dry state.

“Absorbent particulate polymer material area” as used herein refers to the area of the core wherein the first substrate 64 and second substrate 72 are separated by a multiplicity of superabsorbent particles. In FIG. 8, the boundary of the absorbent particulate polymer material area is defined by the perimeter of the overlapping circles. There may be some extraneous superabsorbent particles outside of this perimeter between the first substrate 64 and second substrate 72.

As used herein the term “activation” means any process by which tensile strain produced by intermeshing teeth and grooves causes intermediate web sections to stretch or extend. Such processes have been found useful in the production of many articles including breathable films, stretch composites, apertured materials and textured materials. For nonwoven webs, the stretching can cause fiber reorientation, change in fiber denier and/or cross section, a reduction in basis weight, and/or controlled fiber destruction in the intermediate web sections. For example, a common activation method is the process known in the art as ring rolling.

“Airfelt” is used herein to refer to comminuted wood pulp, which is a form of cellulosic fiber.

The term “basis weight” as used herein refers to the mass of dry fibrous material per unit area, i.e. the mass of dry sheet per unit area, e.g. gram per square meter (gsm).

The term “body facing surface” and “body facing side” refer to surfaces of absorbent articles and/or components thereof which face a wearer's body when the absorbent articles are worn, and the term “garment facing surface” and “garment facing side” refer to surfaces of absorbent articles and/or components thereof that face away from a wearer's body when the absorbent articles are worn. Absorbent articles and components thereof, including the topsheet, backsheet, absorbent core, and any individual materials of their components, have a body facing surface and/or side and a garment facing surface and/or side.

As used herein, the term “bicomponent fibers” refers to fibers which have been formed from at least two different polymers extruded from separate extruders but spun together to form one fiber. Bicomponent fibers are also sometimes referred to as conjugate fibers or multicomponent fibers. The polymers are arranged in substantially constantly positioned distinct zones across the cross-section of the bicomponent fibers and extend continuously along the length of the bicomponent fibers. The configuration of such a bicomponent fiber may be, for example, a sheath/core arrangement wherein one polymer is surrounded by another, or may be a side-by-side arrangement, a pie arrangement, or an “islands-in-the-sea” arrangement.

“Comprise,” “comprising,” and “comprises” are open ended terms, each specifies the presence of what follows, e.g., a component, but does not preclude the presence of other features, e.g., elements, steps, components known in the art, or disclosed herein.

“Consisting essentially of” is used herein to limit the scope of subject matter, such as that in a claim, to the specified materials or steps and those that do not materially affect the basic and novel characteristics of the subject matter.

“Continuous” refers to a bonding pattern including bonding areas that extend continuously along at least one path without a break or interruption. That is, one can trace along the entirety of the continuous bonding pattern without ever having to cross a break or interruption in the pattern. Continuous bonding patterns may be substantially free or free of transition areas interconnecting bonded and non-bonded areas.

“Disposable” is used in its ordinary sense to mean an article that is disposed or discarded after a limited number of usage events over varying lengths of time, for example, less than about 20 events, less than about 10 events, less than about 5 events, or less than about 2 events.

“Diaper” refers to an absorbent article generally worn by infants and incontinent persons about the lower torso so as to encircle the waist and legs of the wearer and that is specifically adapted to receive and contain urinary and fecal waste. As used herein, term “diaper” also includes a “pant” which is defined below.

“Fiber” and “filament” are used interchangeably.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

“Longitudinal” means a direction running substantially perpendicular from a waist edge to a longitudinally opposing waist edge of an absorbent article when the article is in a flat out, uncontracted state, or from a waist edge to the bottom of the crotch, i.e. the fold line, in a bi-folded article. Directions within 45 degrees of the longitudinal direction are considered to be “longitudinal.” “Lateral” refers to a direction running from a longitudinally extending side edge to a laterally opposing longitudinally extending side edge of an article and generally at a right angle to the longitudinal direction. Directions within 45 degrees of the lateral direction are considered to be “lateral.”

The term “machine direction” (MD) is used herein to refer to the direction of material flow through a process. In addition, relative placement and movement of material can be described as flowing in the machine direction through a process from upstream in the process to downstream in the process. The term “cross direction” (CD) is used herein to refer to a direction that is generally perpendicular to the machine direction.

A “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding. The basis weight of nonwoven fabrics is usually expressed in grams per square meter (gsm).

“Pant” or “training pant”, as used herein, refer to disposable garments having a waist opening and leg openings designed for infant or adult wearers. A pant may be placed in position on the wearer by inserting the wearer's legs into the leg openings and sliding the pant into position about a wearer's lower torso. A pant may be pre-formed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the terms “pant” or “pants” are used herein, pants are also commonly referred to as “closed diapers,” “prefastened diapers,” “pull-on diapers,” “training pants,” and “diaper-pants”. Suitable pants are disclosed in U.S. Pat. No. 5,246,433, issued to Hasse, et al. on Sep. 21, 1993; U.S. Pat. No. 5,569,234, issued to Buell et al. on Oct. 29, 1996; U.S. Pat. No. 6,120,487, issued to Ashton on Sep. 19, 2000; U.S. Pat. No. 6,120,489, issued to Johnson et al. on Sep. 19, 2000; U.S. Pat. No. 4,940,464, issued to Van Gompel et al. on Jul. 10, 1990; U.S. Pat. No. 5,092,861, issued to Nomura et al. on Mar. 3, 1992; U.S. Patent Publication No. 2003/0233082 A1, entitled “Highly Flexible And Low Deformation Fastening Device”, filed on Jun. 13, 2002; U.S. Pat. No. 5,897,545, issued to Kline et al. on Apr. 27, 1999; U.S. Pat. No. 5,957,908, issued to Kline et al on Sep. 28, 1999.

“Substantially cellulose free” is used herein to describe an article, such as an absorbent core, that contains less than 10% by weight cellulosic fibers, less than 5% cellulosic fibers, less than 1% cellulosic fibers, no cellulosic fibers, or no more than an immaterial amount of cellulosic fibers. An immaterial amount of cellulosic material would not materially affect the thinness, flexibility, or absorbency of an absorbent core.

“Substantially continuously distributed” as used herein indicates that within the absorbent particulate polymer material area, the first substrate 64 and second substrate 72 are separated by a multiplicity of superabsorbent particles. It is recognized that there may be minor incidental contact areas between the first substrate 64 and second substrate 72 within the absorbent particulate polymer material area. Incidental contact areas between the first substrate 64 and second substrate 72 may be intentional or unintentional (e.g. manufacturing artifacts) but do not form geometries such as pillows, pockets, tubes, quilted patterns and the like.

“Thermoplastic adhesive material” as used herein is understood to comprise a polymer composition from which fibers are formed and applied to the superabsorbent material with the intent to immobilize the superabsorbent material in both the dry and wet state. The thermoplastic adhesive material of the present disclosure forms a fibrous network over the superabsorbent material.

“Thickness” and “caliper” are used herein interchangeably.

In some embodiments, the absorbent article is a diaper. For convenience, an exemplary absorbent article will be described using a diaper as a reference. The skilled person will appreciate that other absorbent articles can also be assembled with topsheets and associated components as disclosed herein.

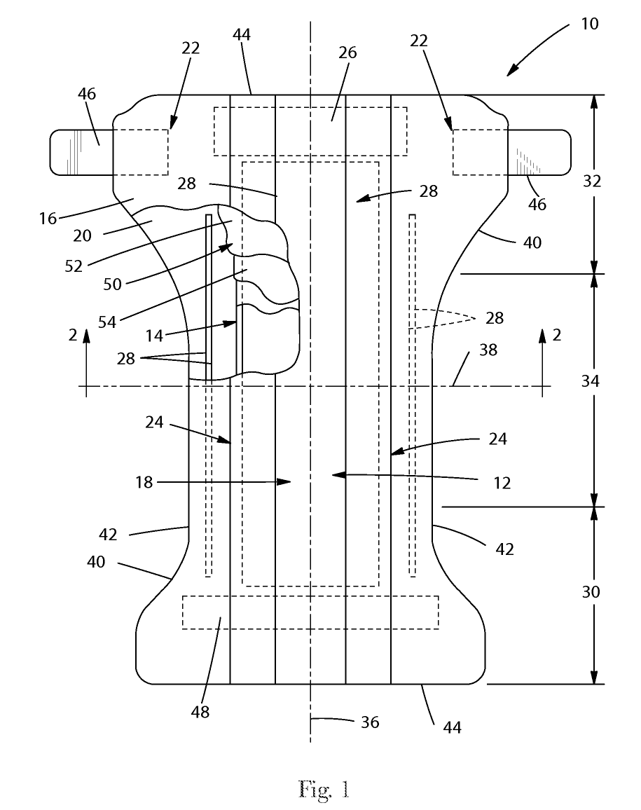

FIG. 1 is a plan view of a diaper 10, shown in a flat out, uncontracted state (i.e., without elastic induced contraction) and with portions of the diaper 10 are cut away to more clearly show the underlying structure of the diaper 10. A portion of the diaper 10 that contacts a wearer is facing the viewer in FIG. 1. The diaper 10 generally may include a chassis 12 and an absorbent core 14 disposed in the chassis.

The chassis 12 of the diaper 10 in FIG. 1 may include an outer covering 16 including a topsheet 18, which may be liquid pervious, and/or a backsheet 20, which may be liquid impervious. The absorbent core 14 may be encased between the topsheet 18 and the backsheet 20. The chassis 12 may also include side panels 22, elasticized leg cuffs 24, and an elastic waist feature 26.

The leg cuffs 24 and the elastic waist feature 26 may each include elastic members 28. One end portion of the diaper 10 may be configured as a first waist region 30 of the diaper 10. An opposite end portion of the diaper 10 may be configured as a second waist region 32 of the diaper 10. An intermediate portion of the diaper 10 may be configured as a crotch region 34, which extends longitudinally between the first and second waist regions 30 and 32. The waist regions 30 and 32 may include elastic elements such that they gather about the waist of the wearer to provide improved fit and containment (elastic waist feature 26). The crotch region 34 is that portion of the diaper 10 which, when the diaper 10 is worn, is generally positioned between the wearer's legs.

The diaper 10 is depicted in FIG. 1 with a longitudinal axis 36 and a transverse axis 38. The periphery 40 of the diaper 10 is defined by the outer edges of the diaper 10 in which the longitudinal edges 42 run generally parallel to the longitudinal axis 36 of the diaper 10 and the end edges 44 run between the longitudinal edges 42 generally parallel to the transverse axis 38 of the diaper 10. The chassis 12 may also comprise a fastening system, which may include at least one fastening member 46 and at least one stored landing zone 48.

The diaper 20 may also include such other features as are known in the art including front and rear ear panels, waist cap features, elastics and the like to provide better fit, as well as containment and aesthetic characteristics. Such additional features are described, for example, in U.S. Pat. Nos. 3,860,003 and 5,151,092.

In order to keep the diaper 10 in place about the wearer, at least a portion of the first waist region 30 may be attached by the fastening member 46 to at least a portion of the second waist region 32 to form leg opening(s) and an article waist opening. When fastened, the fastening system carries a tensile load around the article waist. The fastening system may allow an article user to hold one element of the fastening system, such as the fastening member 46, and connect the first waist region 30 to the second waist region 32 in at least two places. This may be achieved through manipulation of bond strengths between the fastening device elements.

In some embodiments, the diaper 10 may be provided with a re-closable fastening system or may alternatively be provided in the form of a pant-type diaper. When the absorbent article is a diaper, it may include a re-closable fastening system joined to the chassis for securing the diaper to a wearer. When the absorbent article is a pant-type diaper, the article may include at least two side panels joined to the chassis and to each other to form a pant. The fastening system and any component thereof may include any material suitable for such a use, including but not limited to plastics, films, foams, nonwoven, woven, paper, laminates, fiber reinforced plastics and the like, or combinations thereof. In some embodiments, the materials making up the fastening device may be flexible. The flexibility may allow the fastening system to conform to the shape of the body and thus, reduce the likelihood that the fastening system will irritate or injure the wearer's skin.

It is to be appreciated that the topsheet 18, the backsheet 20, and the absorbent core 14 may be assembled in a variety of configurations, such as for example as described generally in U.S. Pat. Nos. 5,554,145; 5,569,234; and 6,004,306. Processes for assembling the diaper include conventional techniques known in the art for constructing and configuring disposable absorbent articles. For example, the backsheet and/or the topsheet can be joined to the absorbent core or to each other by a uniform continuous layer of adhesive, a patterned layer of adhesive, or an array of separate lines, spirals, or spots of adhesive. Adhesives which have been found to be satisfactory are manufactured by H. B. Fuller Company of St. Paul, Minn. under the designation HL-1258 or H-2031.

In some embodiments, the topsheet 18 in FIG. 1 may be fully or partially elasticized or may be foreshortened to provide a void space between the topsheet 18 and the absorbent core 14. Exemplary structures including elasticized or foreshortened topsheets are described in more detail in U.S. Pat. Nos. 5,037,416 and 5,269,775. More description of the topsheets of the present disclosure is found below.

The backsheet 26 may be joined with the topsheet 18. The backsheet 20 may prevent the exudates absorbed by the absorbent core 14 and contained within the diaper 10 from soiling other external articles that may contact the diaper 10, such as bed sheets and undergarments. In certain embodiments, the backsheet 26 may be substantially impervious to liquids (e.g., urine) and comprise a laminate of a nonwoven and a thin plastic film such as a thermoplastic film having a thickness of about 0.012 mm (0.5 mil) to about 0.051 mm (2.0 mils). Suitable backsheet films include those manufactured by Tredegar Industries Inc. of Terre Haute, Ind. and sold under the trade names X15306, X10962, and X10964. Other suitable backsheet materials may include breathable materials that permit vapors to escape from the diaper 10 while still preventing liquid exudates from passing through the backsheet 10. Exemplary breathable materials may include materials such as woven webs, nonwoven webs, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Application No. WO 95/16746, published on Jun. 22, 1995 in the name of E. I. DuPont. Other breathable backsheets including nonwoven webs and apertured formed films are described in U.S. Pat. No. 5,571,096.

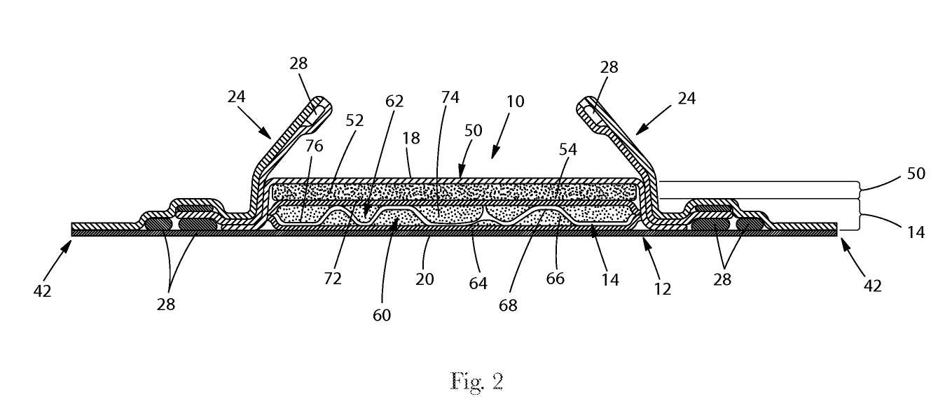

FIG. 2 is a cross sectional view of the diaper in FIG. 1 taken along the line 2-2. As shown in FIG. 2, the topsheet 18 may define an inner, body facing surface, and the backsheet may define an outer, garment facing surface of the diaper 10. And the absorbent core 14 may be positioned between the topsheet and the backsheet. The diaper 10 may also include an acquisition system 50 disposed between the liquid permeable topsheet 18 and a wearer facing side of the absorbent core 14. The acquisition system 50 may be in direct contact with the absorbent core. The acquisition system 50 (also referred to herein as a liquid acquisition layer 50) may comprise a single layer or multiple layers, such as an upper acquisition layer 52 (also referred to herein as a first acquisition layer 52) facing towards the wearer's skin and a lower acquisition layer 54 (also referred to herein as a second acquisition layer 54) facing the garment of the wearer. In some embodiments, the acquisition system 50 may function to receive a surge of liquid, such as a gush of urine. In other words, the acquisition system 50 may serve as a temporary reservoir for liquid until the absorbent core 14 can absorb the liquid.

In some embodiments, the acquisition system 50 may include chemically cross-linked cellulosic fibers. Such cross-linked cellulosic fibers may have desirable absorbency properties. Exemplary chemically cross-linked cellulosic fibers are disclosed in U.S. Pat. No. 5,137,537. In certain embodiments, the chemically cross-linked cellulosic fibers are cross-linked with between 0.5 mole % and 10.0 mole % of a C₂ to C₉ polycarboxylic cross-linking agent or between 1.5 mole % and 6.0 mole % of a C₂ to C₉ polycarboxylic cross-linking agent based on glucose unit. Citric acid is an exemplary cross-linking agent. In some embodiments, polyacrylic acids may be used. Further, according to some embodiments, the cross-linked cellulosic fibers have a water retention value of 25 to 60, or 28 to 50, or 30 to 45. A method for determining water retention value is disclosed in U.S. Pat. No. 5,137,537. In some embodiments, the cross-linked cellulosic fibers may be crimped, twisted, or curled, or a combination thereof including crimped, twisted, and curled.

In some embodiments, one or both of the upper acquisition layer 52 and lower acquisition layer 54 may include a nonwoven, which may be hydrophilic. Further, according to some embodiments, one or both of the upper acquisition layer 52 and lower acquisition layer 54 may comprise the chemically cross-linked cellulosic fibers, which may or may not form part of a nonwoven material. In some embodiments, the upper acquisition layer 52 may comprise a nonwoven, without the cross-linked cellulosic fibers, and the lower acquisition layer 54 may comprise the chemically cross-linked cellulosic fibers. Further, in some embodiments, the lower acquisition layer 54 may comprise the chemically cross-linked cellulosic fibers mixed with other fibers such as natural or synthetic polymeric fibers. According to some embodiments, such other natural or synthetic polymeric fibers may include high surface area fibers, thermoplastic binding fibers, polyethylene fibers, polypropylene fibers, PET fibers, rayon fibers, lyocell fibers, and mixtures thereof. In some embodiments, the lower acquisition layer 54 has a total dry weight, the cross-linked cellulosic fibers are present on a dry weight basis in the upper acquisition layer in an amount from 30% to 95% by weight of the lower acquisition layer 54, and the other natural or synthetic polymeric fibers are present on a dry weight basis in the lower acquisition layer 54 in an amount from 70% to 5% by weight of the lower acquisition layer 54. According to some embodiments, the cross-linked cellulosic fibers are present on a dry weight basis in the first acquisition layer in an amount from 80% to 90% by weight of the lower acquisition layer 54, and the other natural or synthetic polymeric fibers are present on a dry weight basis in the lower acquisition layer 54 in an amount from 20% to 10% by weight of the lower acquisition layer 54.

For example, in some embodiments, the lower acquisition layer 54 may comprise 70% by weight of chemically cross-linked cellulose fibers, 10% by weight polyester (PET), and 20% by weight untreated pulp fibers. According to a second embodiment, the lower acquisition layer 54 may comprise 70% by weight chemically cross-linked cellulose fibers, 20% by weight lyocell fibers, and 10% by weight PET fibers. According to a third embodiment, the lower acquisition layer 54 may comprise 68% by weight chemically cross-linked cellulose fibers, 16% by weight untreated pulp fibers, and 16% by weight PET fibers. In one embodiment, the lower acquisition layer 54 may comprise from 90-100% by weight chemically cross-linked cellulose fibers.

Suitable nonwoven materials for the upper acquisition layer 52 and lower acquisition layer 54 include, but are not limited to SMS material, comprising a spunbonded, a melt-blown and a further spunbonded layer. In certain embodiments, permanently hydrophilic nonwovens, and in particular, nonwovens with durably hydrophilic coatings are desirable. Another suitable embodiment comprises a SMMS-structure. In certain embodiments, the nonwovens are porous.

In certain embodiments, suitable nonwoven materials may include, but are not limited to synthetic fibers, such as PE, PET, and PP. As polymers used for nonwoven production may be inherently hydrophobic, they may be coated with hydrophilic coatings. One way to produce nonwovens with durably hydrophilic coatings, is via applying a hydrophilic monomer and a radical polymerization initiator onto the nonwoven, and conducting a polymerization activated via UV light resulting in monomer chemically bound to the surface of the nonwoven as described in co-pending U.S. Patent Publication No. 2005/0159720. Another way to produce nonwovens with durably hydrophilic coatings is to coat the nonwoven with hydrophilic nanoparticles as described in U.S. Pat. No. 7,112,621 and in PCT Publication No. WO 02/064877.

Nanoparticles may have a largest dimension of below 750 nm. Nanoparticles with sizes ranging from 2 to 750 nm may be economically produced. Some nanoparticles can be easily dispersed in water solution to enable coating application onto the nonwoven, form transparent coatings, and the coatings applied from water solutions are may be sufficiently durable to exposure to water. Nanoparticles can be organic or inorganic, synthetic or natural. Inorganic nanoparticles generally exist as oxides, silicates, and/or, carbonates. Typical examples of suitable nanoparticles are layered clay minerals (e.g., LAPONITE™ from Southern Clay Products, Inc. (USA), and Boehmite alumina (e.g., Disperal P2™ from North American Sasol. Inc.). According to a certain embodiment, a suitable nanoparticle coated nonwoven is that disclosed in the U.S. Patent Publication No. 2004/0158212A1.

Other nonwovens are described in U.S. Pat. Nos. 6,645,569; 6,863,933; and 7,112,621 as well as U.S. Patent Publication Nos. 2003/0148684A1 and 2005/0008839A1.

In some cases, the nonwoven surface can be pre-treated with high energy treatment (corona, plasma) prior to application of nanoparticle coatings. High energy pre-treatment typically temporarily increases the surface energy of a low surface energy surface (such as PP) and thus enables better wetting of a nonwoven by the nanoparticle dispersion in water.

Permanently hydrophilic nonwovens may be used in other parts of an absorbent article. For example, in some embodiments, topsheets and absorbent core layers comprising permanently hydrophilic nonwovens as described above can be used.

According to some embodiments, the upper acquisition layer 52 may include a material that provides recovery when external pressure is applied and removed. Further, according to some embodiments, the upper acquisition layer 52 may comprise a blend of different fibers selected, for example from the types of polymeric fibers described above. In some embodiments, at least a portion of the fibers may exhibit a spiral-crimp which has a helical shape. In some embodiments, the upper acquisition layer 52 may comprise fibers having different degrees or types of crimping, or both. For example, embodiments may include a mixture of fibers having about 8 to about 12 crimps per inch (cpi) or about 9 to about 10 cpi, and other fibers having about 4 to about 8 cpi or about 5 to about 7 cpi. Different types of crimps include, but are not limited to a 2D crimp or “flat crimp” and a 3D or spiral-crimp. According to some embodiments, the fibers may include bi-component fibers, which are individual fibers each comprising different materials, usually a first and a second polymeric material.

The upper acquisition layer 52 may be stabilized by a latex binder, for example a styrene-butadiene latex binder (SB latex), in a certain embodiment. Processes for obtaining such lattices are known, for example, from EP Patent Publication No. EP 0149880A2 and U.S. Patent Publication No. 2003/0105190. In some embodiments, the binder may be present in the upper acquisition layer 52 in excess of about 12%, about 14% or about 16% by weight. For certain embodiments, SB latex is available under the trade name GENFLO™ 3160 (OMNOVA Solutions Inc.; Akron, Ohio).

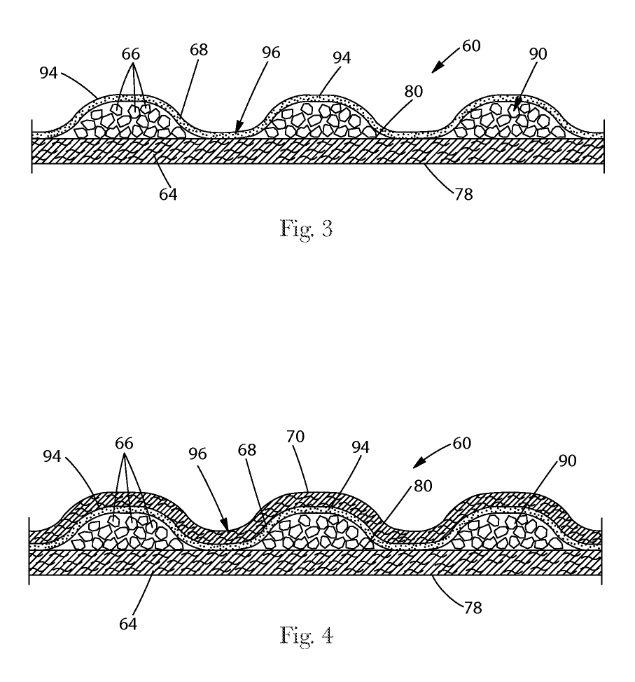

The absorbent core 14, such as shown in FIGS. 1-4, 5A, and 5B, may be disposed between the topsheet 18 and the backsheet 20 and may include two layers, a first absorbent layer 60 and a second absorbent layer 62. As shown in FIG. 3, the first absorbent layer 60 of the absorbent core 14 may include a substrate 64, an absorbent particular polymer material 66 on the substrate 64, and a thermoplastic composition 68 on the absorbent particulate polymer material 66 and at least portions of the first substrate 64 as an adhesive for covering and immobilizing the absorbent particulate polymer material 66 on the first substrate 64. According to another embodiment illustrated in FIG. 4, the first absorbent layer 60 of the absorbent core 14 may also include a cover layer 70 on the thermoplastic composition 68.

As shown in FIG. 2, the second absorbent layer 62 of the absorbent core 14 may also include a substrate 72, an absorbent particulate polymer material 74 on the second substrate 72, and a thermoplastic composition 66 on the absorbent particulate polymer material 74 and at least a portion of the second substrate 72 for immobilizing the absorbent particulate polymer material 74 on the second substrate 72. Although not illustrated, the second absorbent layer 62 may also include a cover layer such as the cover layer 70 illustrated in FIG. 4.

The substrate 64 of the first absorbent layer 60 may be referred to as a dusting layer and has a first surface 78 which faces the backsheet 20 of the diaper 10 and a second surface 80 which faces the absorbent particulate polymer material 66. The substrate 72 of the second absorbent layer 62 may be referred to as a core cover and has a first surface 82 facing the topsheet 18 of the diaper 10 and a second surface 84 facing the absorbent particulate polymer material 74. The first and second substrates 64 and 72 may be adhered to one another with adhesive about the periphery to form an envelope about the absorbent particulate polymer materials 66 and 74 to hold the absorbent particulate polymer material 66 and 74 within the absorbent core 14.

In some embodiments, the substrates 64 and 72 of the first and second absorbent layers 60 and 62 may be a nonwoven material, such as those nonwoven materials described above. In some embodiments, the nonwovens are porous and may have a pore size of about 32 microns.

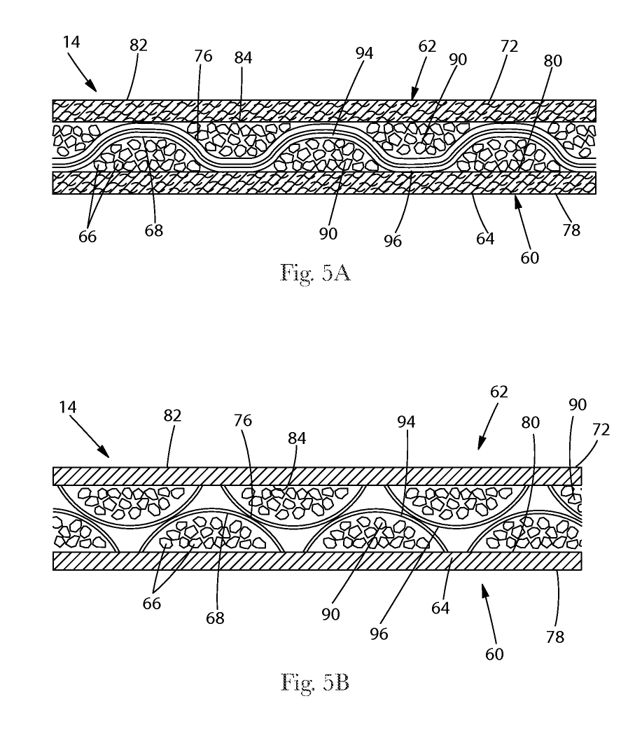

As shown in FIGS. 5A, and 5B, the first and second layers 60 and 62 may be combined to form the absorbent core 14. The absorbent core 14 has an absorbent particulate polymer material area (not shown). The extent and shape of the absorbent particulate polymer material area may vary depending on the desired application of the absorbent core 14 and the particular absorbent article in which it may be incorporated. In some embodiments, the absorbent particulate polymer material area extends substantially entirely across the absorbent core 14. In some embodiments, absorbent particulate polymer material 66 and 74 is substantially continuously distributed across the absorbent particulate polymer material area.

The amount of absorbent particulate polymer material 66 and 74 present in the absorbent core 14 may vary, but in certain embodiments, is present in the absorbent core in an amount greater than about 80% by weight of the absorbent core, or greater than about 85% by weight of the absorbent core, or greater than about 90% by weight of the absorbent core, or greater than about 95% by weight of the core. In some embodiments, the absorbent core 14 consists essentially of the first and second substrates 64 and 72, the absorbent particulate polymer material 66 and 74, and the thermoplastic adhesive composition 68 and 76. In some embodiments, the absorbent core 14 may be substantially cellulose free.

The absorbent particulate polymer material area may have a relatively narrow width in the crotch area of the absorbent article for increased wearing comfort. Hence, the absorbent particulate polymer material area may have a width as measured along a transverse line which is positioned at equal distance to the front edge and the rear edge of the absorbent article, which is less than about 100 mm, 90 mm, 80 mm, 70 mm, 60 mm or even less than about 50 mm.

It some absorbent articles, such as diapers, liquid discharge from the wearer may occur predominately in the front half of the diaper. The front half of the absorbent core 14 may therefore comprise most of the absorbent capacity of the core. Thus, according to certain embodiments, the front half of said absorbent core 14 may comprise more than about 60% of the superabsorbent material, or more than about 65%, 70%, 75%, 80%, 85%, or 90% of the superabsorbent material.

In certain embodiments, the absorbent core 14 may further comprise any absorbent material that is generally compressible, conformable, non-irritating to the wearer's skin, and capable of absorbing and retaining liquids such as urine and other certain body exudates. In such embodiments, the absorbent core 14 may comprise a wide variety of liquid-absorbent materials commonly used in disposable diapers and other absorbent articles such as comminuted wood pulp, which is generally referred to as airfelt, creped cellulose wadding, melt blown polymers, including co-form, chemically stiffened, modified or cross-linked cellulosic fibers, tissue, including tissue wraps and tissue laminates, absorbent foams, absorbent sponges, or any other known absorbent material or combinations of materials. The absorbent core 14 may further comprise minor amounts (typically less than about 10%) of materials, such as adhesives, waxes, oils and the like. Exemplary absorbent structures for use as the absorbent assemblies are described in U.S. Pat. Nos. 4,610,678; 4,834,735; 4,888,231; 5,260,345; 5,387,207; 5,397,316; and 5,625,222.

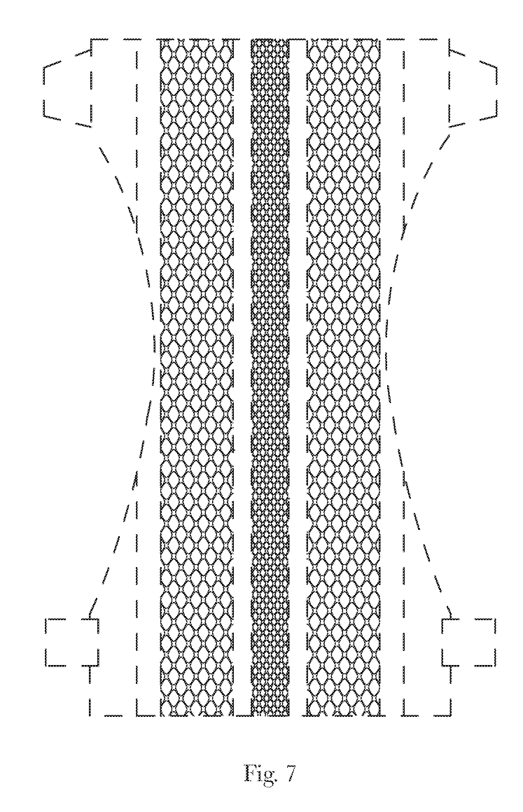

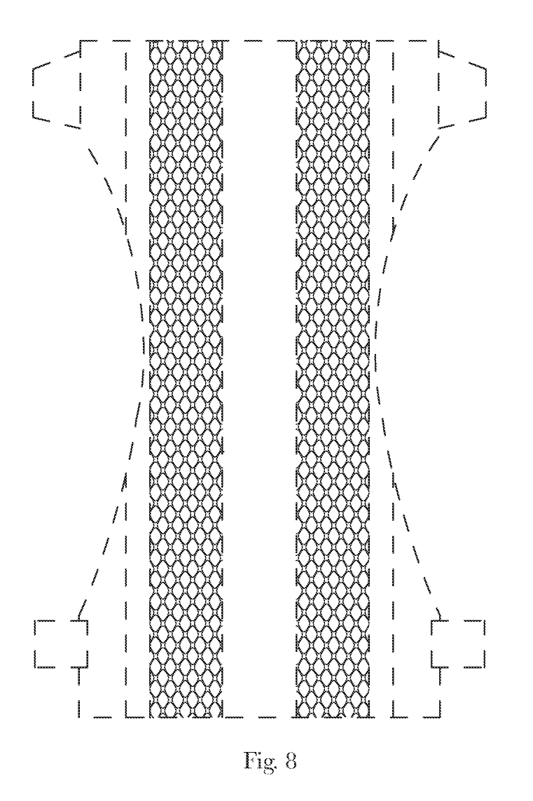

The thermoplastic adhesive material 68 and 76 may serve to cover and at least partially immobilize the absorbent particulate polymer material 66 and 74. In some embodiments, the thermoplastic adhesive material 68 and 76 can be disposed essentially uniformly within the absorbent particulate polymer material 66 and 74, between the polymers. In some embodiments, the thermoplastic adhesive material 68 and 76 may be provided as a fibrous layer which is at least partially in contact with the absorbent particulate polymer material 66 and 74 and partially in contact with the substrate layers 64 and 72 of the first and second absorbent layers 60 and 62. FIGS. 3, 4, and 7 show such a structure wherein the absorbent particulate polymer material 66 and 74 is provided as a discontinuous layer, and a layer of fibrous thermoplastic adhesive material 68 and 76 is laid down onto the layer of absorbent particulate polymer material 66 and 74, such that the thermoplastic adhesive material 68 and 76 is in direct contact with the absorbent particulate polymer material 66 and 74, but also in direct contact with the second surfaces 80 and 84 of the substrates 64 and 72, where the substrates are not covered by the absorbent particulate polymer material 66 and 74. This imparts an essentially three-dimensional structure to the fibrous layer of thermoplastic adhesive material 68 and 76, which in itself is essentially a two-dimensional structure of relatively small thickness, as compared to the dimension in length and width directions. In other words, the thermoplastic adhesive material 68 and 76 undulates between the absorbent particulate polymer material 68 and 76 and the second surfaces of the substrates 64 and 72.

Thereby, the thermoplastic adhesive material 68 and 76 may provide cavities to cover the absorbent particulate polymer material 66 and 74, and thereby immobilizes this material. In a further aspect, the thermoplastic adhesive material 68 and 76 bonds to the substrates 64 and 72 and thus affixes the absorbent particulate polymer material 66 and 74 to the substrates 64 and 72. Thus, in accordance with certain embodiments, the thermoplastic adhesive material 68 and 76 immobilizes the absorbent particulate polymer material 66 and 74 when wet. Some thermoplastic adhesive materials will also penetrate into both the absorbent particulate polymer material 66 and 74 and the substrates 64 and 72, thus providing for further immobilization and affixation. Of course, while the thermoplastic adhesive materials disclosed herein provide a much improved wet immobilization (i.e., immobilization of absorbent material when the article is wet or at least partially loaded), these thermoplastic adhesive materials may also provide a very good immobilization of absorbent material when the absorbent core 14 is dry. The thermoplastic adhesive material 68 and 76 may also be referred to as a hot melt adhesive.

In accordance with certain embodiments, the thermoplastic adhesive material 68 and 76 may comprise, in its entirety, a single thermoplastic polymer or a blend of thermoplastic polymers, having a softening point, as determined by the ASTM Method D-36-95 “Ring and Ball”, in the range between 50° C. and 300° C., or alternatively the thermoplastic adhesive material may be a hot melt adhesive comprising at least one thermoplastic polymer in combination with other thermoplastic diluents such as tackifying resins, plasticizers and additives such as antioxidants. In certain embodiments, the thermoplastic polymer may have a molecular weight (Mw) of more than 10,000 and a glass transition temperature (Tg) usually below room temperature or −6° C.>Tg<16° C. In certain embodiments, typical concentrations of the polymer in a hot melt are in the range of about 20 to about 40% by weight. In certain embodiments, thermoplastic polymers may be water insensitive. Exemplary polymers are (styrenic) block copolymers including A-B-A triblock structures, A-B diblock structures and (A-B)ₙ radial block copolymer structures wherein the A blocks are non-elastomeric polymer blocks, typically comprising polystyrene, and the B blocks are unsaturated conjugated diene or (partly) hydrogenated versions of such. The B block is typically isoprene, butadiene, ethylene/butylene (hydrogenated butadiene), ethylene/propylene (hydrogenated isoprene), and mixtures thereof.

Other suitable thermoplastic polymers that may be employed are metallocene polyolefins, which are ethylene polymers prepared using single-site or metallocene catalysts. Therein, at least one comonomer can be polymerized with ethylene to make a copolymer, terpolymer or higher order polymer. Also applicable are amorphous polyolefins or amorphous polyalphaolefins (APAO) which are homopolymers, copolymers or terpolymers of C₂ to C₈ alpha olefins.

In exemplary embodiments, the tackifying resin may have a Mw below 5,000 and a T_{g} usually above room temperature, typical concentrations of the resin in a hot melt are in the range of about 30 to about 60%, and the plasticizer has a low Mw of typically less than 1,000 and a T_{g} below room temperature, with a typical concentration of about 0 to about 15%.

In certain embodiments, the thermoplastic adhesive material 68 and 76 is present in the form of fibers. In some embodiments, the fibers may have an average thickness of about 1 to about 50 micrometers or about 1 to about 35 micrometers and an average length of about 5 mm to about 50 mm or about 5 mm to about 30 mm. To improve the adhesion of the thermoplastic adhesive material 68 and 76 to the substrates 64 and 72 or to any other layer, in particular any other nonwoven layer, such layers may be pre-treated with an auxiliary adhesive.

The absorbent core 14 may also comprise an auxiliary adhesive which is not illustrated in the figures. The auxiliary adhesive may be deposited on the first and second substrates 64 and 72 of the respective first and second absorbent layers 60 and 62 before application of the absorbent particulate polymer material 66 and 74 for enhancing adhesion of the absorbent particulate polymer materials 66 and 74 and the thermoplastic adhesive material 68 and 76 to the respective substrates 64 and 72. The auxiliary glue may also aid in immobilizing the absorbent particulate polymer material 66 and 74 and may comprise the same thermoplastic adhesive material as described hereinabove or may also comprise other adhesives including but not limited to sprayable hot melt adhesives, such as H.B. Fuller Co. (St. Paul, Minn.) Product No. HL-1620-B. The auxiliary glue may be applied to the substrates 64 and 72 in various ways. For example, in some embodiments, the auxiliary glue may be applied in about 0.5 to about 1 mm wide slots spaced about 0.5 to about 2 mm apart.

The cover layer 70 shown in FIG. 4 may include the same material as the substrates 64 and 72, or may include a different material. In certain embodiments, the materials of the cover layer 70 are the nonwoven materials, such as the materials described above as useful for the substrates 64 and 72.

Although much of the present discussion is presented in the context of absorbent articles in form of diapers, it is to be appreciated that other absorbent articles, such as sanitary napkins can also be assembled with the topsheets and associated components as disclosed herein. Absorbent articles, such as sanitary napkins may be designed to be worn in close proximity to the crotch of the wearer. Such absorbent articles need to provide for fluid acquisition and retention and may look aesthetically pleasing, as well as be comfortable to wear. Examples of sanitary napkins are provided in U.S. Patent Publication Nos. 2010/0036339; 2010/0036347; and 2010/0036349, the disclosures of which are herein incorporated by reference. In use, sanitary napkins are stressed by a variety of fluid handling demands. Given the variety of fluid handling demands placed on different portions of an absorbent article, such as a sanitary napkin, the different physical interactions between portions of an absorbent article and portions of a wearer's body, and different moisture and chemical environments of different portions of a wearer's crotch region, there is continuing and unaddressed need for absorbent articles having aesthetically appealing, are comfortable to wear, but do not compromise the performance of the absorbent article. Sanitary napkins made with the topsheets described herein provide an aesthetically appealing surface to the body facing side of the article while not unduly compromising the performance of the acquisition layer and maintaining the comfort of the article during wearing.

### The Topsheet

As discussed above, the absorbent articles described herein include a topsheet 18. The inventors surprisingly found that absorbent articles including certain topsheets can provide improved actual and/or perceived performance, e.g. absorbency and/or softness. Such topsheets are characterized by having a certain basis weight, activating the topsheet prior to making a continuous bonded pattern on it, where multiple continuous patterns are optionally arranged along the longitudinal axis of the topsheet. The topsheets are optionally, further characterized with pre-bonding, embossing, opacity, and/or coloring.

The topsheet may be a nonwoven material made of synthetic fibers alone or in combination with natural fibers. Examples of natural fibers may include cellulosic natural fibers, such as fibers from hardwood sources, softwood sources, starches or other non-wood plants. The synthetic fibers can be any material, such as, but not limited to, those selected from the group consisting of polyolefins (polypropylene and polypropylene copolymers, polyethylene and polyethylene copolymers), polyesters (e.g., polyethylene terephthalate), polyethers, polyamides, polyesteramides, polyvinylalcohols, polyhydroxyalkanoates, polysaccharides, and combinations thereof. Further, the synthetic fibers can be a single component (i.e. a single synthetic material or a mixture makes up the entire fiber), bi-component (i.e. the fiber is divided into regions, the regions including two or more different synthetic materials or mixtures thereof and may include co-extruded fibers and core and sheath fibers) and combinations thereof. Bi-component fibers can be used as a component fiber of the nonwoven material, and/or they may be present to act as a binder for the other fibers present in the nonwoven material. Any or all of the fibers may be treated before, during, or after manufacture to change any desired properties of the fibers. Preferably, the topsheet comprises at least 20% of synthetic fibers, or at least 30% of synthetic fibers or at least 50% of synthetic fibers. In some embodiments, the topsheet comprises 100% of synthetic fibers. Synthetic fibers are preferably thermoplastic fibers. Preferably, the topsheet is made of a nonwoven material made of a polyolefin, such as polyethylene, polypropylene or mixtures thereof. The topsheet may be a single layer, or a multilayer nonwoven web, i.e. a laminate. The laminate may comprise spunbond layer(s) (S), and/or meltblown layer(s) (M), and/or carded layer(s) (C). Suitable laminates include, but are not limited to, SS, SSS, SMS or SMMS. In some embodiments, the topsheet is a spunbond nonwoven material, such as a mono layer spunbond (S), or a dual layer spunbond (SS) or a nonwoven material comprising more than two layers, such as a spunbond nonwoven with three layers (SSS). In an alternative embodiment, the topsheet is made of a laminate of a nonwoven web and a film. In such laminates, the continuous bonded pattern described herein will be made on the nonwoven prior to lamination of the nonwoven with the film. Except as otherwise noted, the below description of the topsheet shall apply also a nonwoven substrate.

The topsheet or nonwoven, as the case may be, typically has a low basis weight, i.e. from 12 gsm to 18 gsm, or from 13 gsm to 16 gsm, or of 15 gsm, which ranges exhibit excellent fluid handling properties. It is believed that such a topsheet has the capacity to acquire liquid in a desirable way, i.e. the fluid, such as urine, readily penetrates through the thickness of the topsheet. Furthermore, the topsheet does not retain much fluid and thus minimizes the risk of rewet that may be observed when fluids are retained within the topsheet. Minimizing the rewet contributes to maintain the skin of the diaper's wearer in a dry state and thus to increase the comfort of the wearer.

The topsheet is formed by activation, then bonding of a suitable base substrate, namely, a base substrate is (1) permanently deformed through fiber rearrangement and fiber separation and breakage producing permanent fiber dislocation (referred to hereinafter as “fiber displacement”) such that the structured substrate has a thickness value which is higher than that of the base substrate and then, (2) modified by bonding to form a compressed region below the thickness of the base substrate in a continuous pattern. The activation induces fiber displacement and forms a three dimensional texture which increases the fluid acquisition properties of the base substrate. Fiber displacement processes involve permanent mechanical displacement of fibers via rods, pins, buttons, structured screens or belts or other suitable technology. Non-limiting examples include ring-rolling and SELFing. The permanent fiber dislocation provides additional thickness or caliper compared to the base substrate. The additional thickness increases specific volume of the substrate and also increases fluid permeability of the substrate. The bonding is believed to improve the mechanical properties of the base substrate and can enhance the depth of channels in between displaced fiber regions for fluid management. Suitable methods for activating and bonding the topsheet are disclosed in USPA 2010/0310837A1. Processes for bonding fibers are also described in detail in “Nonwovens: Theory, Process, Performance and Testing” by Albin Turbak (Tappi 1997). Typical bonding methods include mechanical entanglement, hydrodynamic entanglement, needle punching, and chemical bonding and/or resin bonding; however, thermal bonding such as thru-air bonding utilizing heat and thermal point bonding utilizing pressure and heat are preferred with thermal point bonding being more preferred.

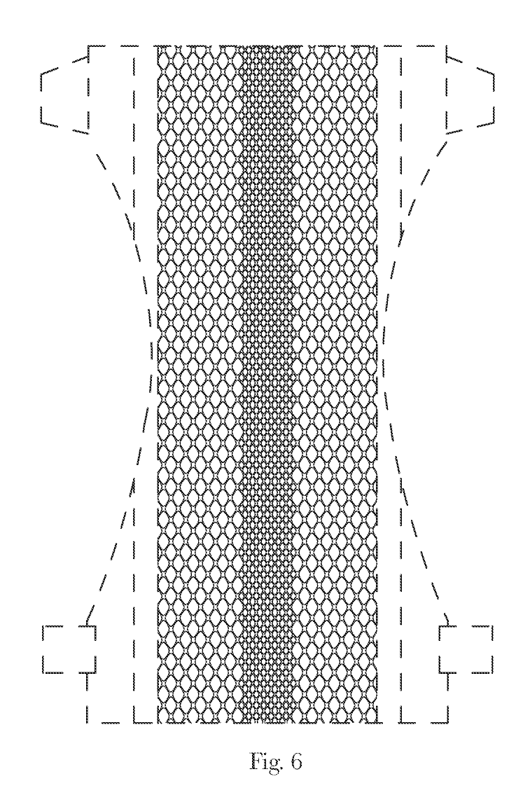

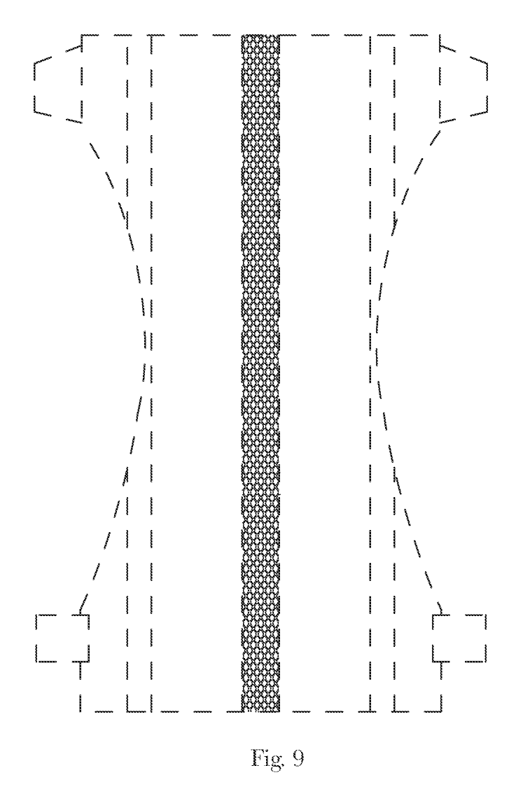

The bonding relies upon heat and pressure to fuse the filaments together in a continuous pattern that is reproducible along the length of the structured substrate so that a repeat pattern can be observed. The bonding may advantageously be done through a pressurized roller nip in which at least one of the rolls is heated, preferably both rolls are heated. If the bonding is done when the base substrate is already heated, then the pressurized roller nip may be heated less or not at all. Examples of patterns of over bond regions 11 are shown in FIGS. 6-9; however, other bond patterns are possible. FIG. 6 shows bond regions 11 forming three continuous patterns in the machine direction, where two of the patterns are identical and the third pattern is similar but of small scale. The total area bonded is preferably from 10 to 60%, or 15 to 45%, or 25 to 35%, of the total area of the topsheet. Without being limited by theory, it is believed that bonding of less than about 10% of the area is insufficient to convey to the viewer of the topsheet, e.g. a caregiver, the actual or perceived product attributes, e.g. softness, as described below. Conversely, it is believed that bonding more than about 60% of the area is too much to convey the attribute, because the area characterized by the actual or perceived quality is not sufficiently distinguishable from the remainder of the topsheet. The narrower ranges disclosed above are believed to represent percent areas that are preferred for such conveyance.

The continuous bonded patterns can take any shapes, such as circular, diamond, triangular, rectangular, square, oval, or any other regular or irregular shapes, provided that the shape can be part of a repeating pattern.

In some embodiments, a plurality of bonded patterns may be presented along a longitudinal axis of the topsheet. For example, different patterns may be presented at the longitudinal periphery of the topsheet compared to the center of topsheet. As used herein “longitudinal periphery of the topsheet” refers to an area of the topsheet starting from a longitudinal edge of the topsheet and extending towards the longitudinal axis, but not reaching the center of the topsheet. Thus, there is a left side longitudinal periphery of the topsheet, a right side longitudinal periphery of the topsheet, and a center region, which includes the longitudinal axis. In some embodiments, the area of the topsheet may be divided equally into thirds, i.e. 1:1:1. In other embodiments, the area of the topsheet may be divided into the following portions, respectively: 3:1:3, 2:1:2, or 1.5:1:1.5. It is believed that such arrangements help communicate to consumers that absorbent activity occurs in central region and that the lateral regions channel fluid into the central region.

The continuous bonded pattern is believed to confer an increased softness to the topsheet whilst not unduly compromising its strength. Additionally, the combination of the patterns along the longitudinal axis enhances the visibility of the pattern on the surface of the topsheet. It is believed that the alignment enhances the appearance of the bonded areas and contributes to enhance the visual distinction of the pattern on the topsheet. Overall, this is believed to increase the attractiveness of the product among consumers and overcome the technical challenge of increasing the visibility of the pattern without compromising the fluid handling properties of the topsheet.

In some embodiments, determining the actual dimensions of the bonded area on the topsheet material itself may be difficult as the change from non-bonded area to bonded area may be gradually. Therefore, for the present invention, the dimensions given herein for the bonded points are generally determined by the dimensions of the equipment used to provide the bonded pattern to the topsheet. Thus, while the dimensions can be approximated on the topsheet material, the pattern provided on the equipment has to be determined and are equated with the bonded area for the present invention. In embodiments, wherein the pattern used to provide the bonded area (such as the protrusions on a bonding roll or calendaring roll) do not have straight side-walls, the largest dimensions (i.e. the largest dimension of the width of the protrusions, not the height of the protrusions) on the equipment are considered to be the dimensions for the present invention.

In some embodiments, the topsheet may optionally be colored. By “colored” as used herein, it is meant colored in a color other white, such as but not limited to pink, yellow, purple, red, orange, blue, green, periwinkle and any declination thereof or mixture thereof. In some embodiments, the colored topsheet is colored in a pale or delicate variation of the color, i.e. the topsheet is tinted.

Colors can be measured according to an internationally recognized 3D solid diagram of colors where all colors that are perceived by the human eye are converted into a numerical code. The system is based on three dimensions (x,y,z) and specifically L, a, b.

When a color is defined according to this system L represents lightness (0=black, 100=white), a and b independently each represent a two color axis, a representing the axis red/green (+a=red, −a=green), while b represents the axis yellow/blue (+b=yellow, −b=blue).

Any color is identified by a unique ΔE value which is mathematically expressed by the equation:

ΔE=[(L_{ref}−Lₛₐₘₚₗₑ)²+(a_{ref}−aₛₐₘₚₗₑ)²+(b_{ref}−bₛₐₘₚₗₑ)²)]^{1/2}

AE represents graphically the distance between the reference color and the no color point (i.e. centre of sphere L_{ref}=50, a_{ref}=0, b_{ref}=0) of the 3D model.

Color can be measured using the colorimeter MINOLTA mode CR-300 instrument (available from the Minolta Company, Japan) which provides the coordinates L, a, b and from which the ΔE value can be determined.

The L Hunter scale values (L), utilized herein to define the darkness/lightness of the materials of the topheet, are units of color measurement in the Hunter Color system. A complete technical description of the system can be found in an article by R.S. Hunter, ‘photoelectric color difference Meter’, Journal of the Optical Society of America, Vol. 48, pp. 985-95, 1958. Devices specially designed for the measurement of color on the Hunter scales are described in U.S. Pat. No. 3,003,388 to Hunter et al., issued Oct. 10, 1961. In general, Hunter Color “L” scale values are units of light reflectance measurement, and the higher the value is, the lighter the color is since a lighter colored material reflects more light. In particular, in the Hunter Color system the “L” scale contains 100 equal units of division, absolute black is at the bottom of the scale (L=0) and absolute white is at the tops of the scale (L=100). Thus in measuring Hunter Color values of the topsheet, the higher the “L” scale value, the lighter the material.

It is to be understood that the L Hunter values and color values ΔE considered herein are those measured on the materials of interest (e.g., topsheet), taken in layer or folded upon itself, so that upon additional layering of the same material or additional folding thereof, the L Hunter value and color value stay constant. Indeed the L Hunter value and ΔE are those of the material per se without any influence of the support onto which the material is disposed in the instrument for its measure. In other words, when measuring the L Hunter value of topsheet, several layers of this material are superposed or a layer thereof is folded upon itself several times before measuring its L Hunter value, the same material is measured again after additional layering or folding, this later operation is performed as needed until upon additional layering or folding the L Hunter value does not change any more. This value is the one to be considered herein.

In some embodiments, the colored topsheet has an L value from at least 60, such as from 60 to 95, or from 70 to 95, or from 80 to 90. A topsheet which is colored such that it fulfills the L Hunter value has a delicate or pale color.

In some embodiments, the colored topsheet has an “a” value from about −50.0 to about +50.0, or from about −30.0 to about +30.0, or from about −20.0 to about +20.0 or from about −10.0 to about +10.0. Furthermore, the colored topsheet has a “b” value from about −50.0 to about +50.0, or from about −35.0 to about +25.0, or from about −25.0 to about +15.0 or from about −20.0 to about +5.0.

In some embodiments where the topsheet is colored, it may be colored all over its surface, typically uniformly colored all over its surface. Preferably, the topsheet is colored with a single color, typically uniformly colored all over its surface with a single color.

Coloring can be done by any methods available in the art. The topsheet may be colored after the manufacturing thereof or alternatively suitable pigments may be added to the material, e.g. polyolefin, out of which the fibers of the nonwoven material are made.

Suitable coloring agent may be generally termed as pigment which refers to an insoluble color matter used in finely dispersed forms. The coloring agents may be dyes, organic pigments or inorganic pigments. Exemplary organic pigments may include: C.I. Pigment Yellow 1, C.I. Pigment Yellow 3, C.I. Pigment Yellow 13, C.I. Pigment Red 5, C.I. Pigment Red 7, C.I. Pigment Red 12, C.I. Pigment Red 112, C.I. Pigment Red 122, C.I. Pigment Blue 1, C.I. Pigment Blue 2, C.I. Pigment Blue 16, C.I. Vat Blue 4, C.I. Vat Blue 6, or Carbon black. Exemplary inorganic pigments may include carbon black (e.g., Pigment Black 7), iron oxides, ferric oxide black (e.g., Pigment Black 11), chromium oxide, or ferric ammonium ferrocyanide. Exemplary dyes may include: Solvent Yellow 14, Dispersed Yellow 23, Metanil Yellow, Solvent Red 111, Dispersed Violet 1, Solvent Blue 56, Solvent Orange 3, Solvent Green 4, Acid Red 52, Basic Red 1, Solvent Orange 63, or Jet Black.

The skilled person will appreciate that topsheet 18 may also be embossed by various apparatuses and methods, provided that such embossing does not substantially negatively affect the actual or perceived performance of the topsheet herein. As used herein, the term “embossed” refers to a densification of the fibers comprised by the embossed area, while the fibers are not bonded to each other by melting them to each other. Embossing is typically achieved by applying pressure. The embossing process may include the application of heat as long as the temperature does not exceed the melting temperature of the fibers.

One way to reduce the bulk of absorbent articles is to optimize the distribution of the absorbent material in order to provide the absorbent material mostly or even only in those regions where it can readily absorb liquid at the expense of providing it in other areas. Another way is to use materials, such as films or nonwoven webs, with relatively low basis weight. However, as the basis weight of films and nonwoven webs reduced, these materials typically become more translucent. Increased translucency is especially apparent in those parts of disposable absorbent articles, where only very few materials are overlaying each other or where even only one layer of material is used. However, many consumers associate translucency with insufficient absorbency and thus, low performance and an increased risk of leakage. Furthermore, “see-through” is believed by some to give the absorbent article a cheap, low-quality appearance. Additionally, in those embodiments where the absorbent core is substantially cellulose free, the resulting absorbent structures have a reduced thickness in the dry state compared to conventional absorbent structure comprising cellulosic fibers. Although the reduced thickness helps to improve the fit and comfort of the absorbent article for the wearer, it may also be perceived by some as lacking sufficient absorbent material and hence, be of low quality. Surprisingly, the topsheets described herein are believed to increase the acceptance of the products among consumers as reinforcing the perception of absorbency and softness.

In some embodiments, the topsheet described herein has an opacity of at least 15%, or at least 17%, or at least 20%, or an opacity of 15% to 30%, according to the test method provided herein. In one embodiment, the opacity of the topsheet is homogeneous throughout the topsheet. If the topsheet material comprises more than one layer of material, than the complete topsheet material, i.e. all layers, are subjected to the opacity test method described below.

To achieve the desired degree of opacity, the topsheet may be tinted. Tinting can be facilitated to provide one or more colored pigments when the topsheet material is manufactured. For example, suitable pigments can be provided to the polyolefin out of which the fibers of a nonwoven web are made. For a given material, opacity also typically increases with increased basis weight. Moreover, the opacity of the topsheet can be increased by adding titanium dioxide (TiO₂), which is a non-colored (white) pigment which enhances the “whiteness” of the topsheet. Titanium dioxide is typically present, if at all, in an amount less than about 5%, by weight, and more typically in an amount of from 0.5% to 5%, by weight, or from 1% to 4%, by weight, or from 2% to 4%, by weight.

The opacity of the topsheet can also be modified by applying lotion onto the wearer facing surface of the topsheet. In some embodiments, a lotion is thus provided on the wearer facing surface of the topsheet. Preferably, the lotion should not decrease the opacity of the topsheet and more preferably, the lotion increases the opacity of the topsheet. In such embodiments, the opacity is determined with the lotion applied onto the topsheet. However, in another embodiment the topsheet is free of lotion applied onto either of its surfaces.

### Test Methods

### Topsheet Dryness Test

The Topsheet Dryness Test determines the amount of liquid retained in the topsheet. This test simulates the in-use performance of diaper. The test should be carried out at about 22+/−2° C. and at 50+/−5% relative humidity.

### Sample Preparation and Loading

The test sample comprises an absorbent core including an acquisition system, a topsheet and a backsheet. The absorbent core including the acquisition system of the present sample is as available in diapers sold under the Tradename Pampers® Active Fit, Size 4, sold in the UK. On the topsheet, a rectangular part (55 mm×105 mm) is marked (centered in cross direction) and the leading side of the 55 mm wide rectangular part is placed 28 mm away from the leading edge of the absorbent core.

The synthetic urine used in these test methods is a 0.9% NaCl solution in de-ionized water.

The test sample is arranged to lie flat on a platform of the equipment with the topsheet facing up. Suitable equipment includes equipment as available from FKV S.r.1, Italy. A plate having a 5 cm diameter opening in its middle is placed on top of the sample on the loading zone of the sample. The loading zone is centered under the 5 cm diameter opening. The loading zone of the sample is defined as the zone located at 102 mm from the leading edge of the absorbent core and centered relative to the longitudinal edges of the absorbent core. A pressure of 2.07 kPa is applied to the sample.

The test sample is loaded with a 75 ml gush of synthetic urine at a rate of 15 ml/s using a pump (Model 7520-00, supplied by Cole Parmer Instruments., Chicago, U.S.A.), from a height of 5 cm above the sample surface. The synthetic urine is introduced to the sample through a cylinder fitted in the 5 cm opening of the plate. Precisely 5 minutes after the gush, the sample is loaded again with a 75 ml gush of synthetic urine at a rate of 15 ml/s. The loading step is performed two more times as described herein at precisely 5 minute gush intervals.

### Measurement

10 minutes after the absorbance of the 4^{th} gush, the marked topsheet rectangle (55 mm×105 mm) is cut from the sample and put into a petri dish. The weight of the topsheet and petri dish is then measured and recorded as the wet weight.

The marked topsheet rectangular part in the petri dish is placed for 12 hours in a 60° C. preheated oven. The weight of the topsheet and petri dish is then measured and recorded as the dry weight.

The difference between the wet and dry weight is specified as the retained liquid in topsheet.

### Rewet Test

The rewet test determines the in use performance of a diaper. It measures the amount of liquid which is released by the topsheet. High rewet properties are not desirable since they would make the wearers uncomfortable. The test should be carried out at about 22+/−2° C. and at 50+/−5% relative humidity.

### Sample Preparation and Loading

The sample is prepared and loaded as described above.

### Measurement

Before executing the test, a collagen film as purchased from NATURIN GmbH, Weinhein, Germany, under the designation of COFFI and at a basis weight of about 28 g/m² is prepared by being cut into sheets of 70 mm diameter e.g. by using a sample cutter device, and by equilibrating the film in the controlled environment of the test room (see above) for at least 12 hours (tweezers are to be used for all handling of the collagen film).

10 minutes after the last gush of the sample load preparation is absorbed, the test sample is carefully placed flat on a lab bench.

4 sheets of the precut and equilibrated collagen material are weighed with at least one milligram accuracy, and then positioned centered onto the loading point of the topheet (i.e. point centered relative to the longitudinal edges of the absorbent core and 102 mm away from the leading edge of the absorbent core) and covered by perspex plate of 90 mm diameter, and about 20 mm thickness. A weight of 9.1 kg is carefully added (also centered). After 30+/−2 seconds the weight and perspex plate are carefully removed again, and the collagen films are reweighed.

The difference between the weight of the dry collagen sheets and the wet collagen sheets after load is the moisture pick up of the collagen film, expressed in mg.

### Opacity Test Method

Opacity is a measure of the capacity of a material to obscure the background behind it. The value for opacity is obtained by dividing the reflectance obtained with a black backing (RB) for the material, by the reflectance obtained for the same material with a white background (WB). This is called the contrast ratio (CR) method.

$$\% Opacity = \frac{R B}{RW} \times 100$$

Using a Hunter Colorimeter set to XYZ color scale, opacity is defined as

$$\% Opacity = \frac{Y reading over black plate}{Y reading over white plate} \times 100$$

### Sample Preparation

A specimen of suitable size (generally about 10 cm square) is cut for analysis. The specimen must be free of creases, wrinkles, tears and other obvious defects.

If the opacity of the material is affected by temperature and/or humidity, the specimens must be conditioned under standard TAPPI conditions (73° F. (22.8° C.); 50% RH) until equilibrium is reached, and measured under those conditions.

If the topsheet material is treated with one or more surfactants, the material used for the test is the surfactant-treated topsheet material.

### Equipment

Hunter Labscan® XE available from Hunter Associates Laboratory, Inc., USA. The instrument is configured as follows:

### Geometry 45°/0°

### Color Scale XYZ

### Illuminant D65

### Observer 10°

The colorimeter is calibrated using the standard gloss black glass and gloss white tile supplied with the instrument according to the manufacturer's instructions.

### Test Procedure

The specimen is placed on the white tile and inserted into the colorimeter according to the manufacturer's instructions. The machine direction of the specimen should be aligned front-to-back in the instrument. The Y reading is recorded to the nearest 0.1 unit. The procedure is repeated using the black standard plate instead of the white standard tile.

Ten specimens are measured and the opacity results averaged to obtain the % opacity value for the material.

$$\% Opacity = \frac{{ }^{``} {Y}^{″} on black plate}{{ }^{``} {Y}^{″} on white plate} \times 100$$

### Examples of Topsheet Materials

### Example 1

A 17 gsm bicomponent nonwoven, activated and bonded with the bond pattern shown in FIG. 6, and having an opacity of 24%.

### Example 2

An 18 gsm nonwoven, labeled HESB P11.

### Example 3

A 15 gsm nonwoven, embossed, white.

### Example 4

A 15 gsm nonwoven, labeled P10, white, bonded but not activated, and having an opacity of 21%

### Example 5

A 15 gsm nonwoven, labeled P120, tinted teal, and embossed.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

1. An absorbent article comprising an activated, then bonded topsheet, wherein said topsheet has a basis weight from about 12 gsm to about 18 gsm, and the topsheet comprises a continuous bonded pattern, the bonded area of the pattern being from about 10% to about 60% of the total surface area of the topsheet.

2. The absorbent article according to claim 1, wherein the bonded area of the bonded pattern is from about 25% to about 35%, of the total surface area of the topsheet.

3. The absorbent article according to claim 1, wherein the topsheet comprises a longitudinal axis and a first longitudinal periphery area starting from a longitudinal edge of the topsheet and extending towards the longitudinal axis but not reaching it, a second longitudinal periphery area starting from another longitudinal edge of the topsheet and extending towards the longitudinal axis but not reaching it, and a center area that includes the longitudinal axis, each of such three areas comprising a continuous bonded pattern.

4. The absorbent article according to claim 3, wherein the two longitudinal areas and the center area are substantially parallel to each other.

5. The absorbent article according to claim 3, wherein the area of the first longitudinal areas to the center area to the second longitudinal area is from about 1:1:1 to about 3:1:3.

6. The absorbent article according to claim 1, wherein the bonded pattern comprises shapes selected from the group consisting of circular, diamond, triangular, rectangular, square, and oval.

7. The absorbent article according to claim 1, wherein the bonded pattern comprises a color that is different than a color of the remainder of the topsheet.

8. The absorbent article according to claim 1, wherein the topsheet further comprises an embossing pattern.

9. The absorbent article according to claim 1, wherein the topsheet has an opacity of at least about 15%.

10. The absorbent article according to claim 9, wherein the topsheet has an opacity of from about 15% to about 30%.

11. The absorbent article according to claim 1, wherein the bonded pattern is formed by thermal bonding.

12. The absorbent article according to claim 1, wherein the topsheet is a nonwoven material comprising fibers selected from the group consisting of synthetic fibers and combinations of synthetic fibers and natural fibers.

13. The absorbent article according to claim 12, wherein the nonwoven material comprises polyethylene, polypropylene, or combinations thereof.

14. An absorbent article comprising an activated, then bonded topsheet, wherein said topsheet has a basis weight of from about 12 gsm to about 18 gsm, the topsheet comprising a longitudinal axis and a first longitudinal periphery area starting from a longitudinal edge of the topsheet and extending towards the longitudinal axis but not reaching it, a second longitudinal periphery area starting from another longitudinal edge of the topsheet and extending towards the longitudinal axis but not reaching it, and a center area that includes the longitudinal axis, each of such three areas comprising a continuous bonded pattern.

15. The absorbent article according to claim 14, wherein the two longitudinal areas and the center area are substantially parallel to each other.

16. The absorbent article according to claim 14, wherein the bonded area of the bonded pattern is from about 15% to about 45%, of the total surface area of the topsheet.

17. A nonwoven substrate comprising:
(a) a plurality of bonded regions, each of said plurality of bonded regions having a continuous bonding pattern comprising a geometric shape cooperatively associated thereto;
(b) a first of said bonded regions comprising a first continuous bonding pattern of a first geometric shape;
(c) a second of said bonded regions comprising a second continuous bonding pattern of a second geometric shape; and,
(d) wherein said first and second bonded regions are disposed adjacent each other within said substrate.

18. The nonwoven substrate according to claim 17, wherein a third of said bonded regions comprises a third continuous bonding pattern of a third geometric shape; and, wherein said second and third bonded regions are disposed adjacent each other within said substrate.

19. The nonwoven substrate according to claim 18, wherein the three bonded regions are substantially parallel to each other.

20. The nonwoven substrate according to claim 17, wherein said nonwoven has a basis weight of from about 12 gsm to about 18 gsm.

21. The nonwoven substrate according to claim 17, wherein the bonded area of the bonding pattern is selected from about 15% to about 45%, of the total surface area of the topsheet.

22. The nonwoven substrate according to claim 17, wherein the area of the first bonded region to the second bonded region to the third bonded region is from about 1:1:1 to about 3:1:3.

23. The nonwoven substrate according to claim 17, wherein the bonding pattern comprises shapes selected from the group consisting of circular, diamond, triangular, rectangular, square, and oval.

24. The nonwoven substrate according to claim 17 wherein the bonding pattern comprises a color that is different than a color of the remainder of the nonwoven.

25. The nonwoven substrate according to claim 17, wherein the nonwoven further comprises an embossing pattern.

26. The nonwoven substrate according to claim 17, wherein the nonwoven has an opacity of at least about 15%.

27. The nonwoven substrate according to claim 26, wherein the nonwoven has an opacity of from about 15% to about 30%.

28. The nonwoven substrate according to claim 17 wherein the bonding pattern is formed by thermal bonding.

29. The nonwoven substrate according to claim 17, wherein the nonwoven comprises fibers selected from the group consisting of synthetic fibers and combinations of synthetic fibers and natural fibers.

30. The nonwoven substrate according to claim 29, wherein the nonwoven material comprises polyethylene, polypropylene, or combinations thereof.

31. The nonwoven substrate according to claim 18 wherein the first and third geometric shapes are the same as each other, but different from the second geometric shape.
